(19) United States
(12) Patent Application Publication
(10) Pub. No.: US 2008/0096926 A1
(43) Pub. Date: Apr. 24, 2008

(54) Treatment Of Cognitive Impairment Using A Selective Dopamine D1 Receptor Agonist

(75) Inventor: Jacques Kusmierek, Paris (FR)

Correspondence Address: FISH & RICHARDSON P.C., PO BOX 1022, MINNEAPOLIS, MN, 55440-1022, US

(73) Assignee: DrugAbuse Sciences SAS, Paris (FR)

(21) Appl. No.: 10/533,051

(22) PCT Filed: Oct. 22, 2003
(86) PCT No.: PCT/US03/33440

§ 371 Date: Sep. 16, 2005

Related U.S. Application Data
(60) Provisional application No. 60/420,582, filed on Oct. 22, 2002.

(51) Int. Cl. A61K 31/451; A61P 25/28

(52) U.S. Cl. 514/327

(57) ABSTRACT

Methods of using DAS-431 under conditions where it is administered at a dose that results in improved cognitive performance but without significant side effects are provided. Symptoms can be ameliorated during the period of treatment and, frequently, for substantially longer periods thereafter. In addition, the DAS-431 is provided at a level which does not negatively affect responsiveness to subsequent doses of DAS-431. Few adverse effects are observed, and the treatments can result in long term relief, so as to avoid frequent repetitive treatment.

### TECHNICAL FIELD

The invention is concerned with providing a patient with a selective dopamine D1-like receptor agonist for the long term treatment of cognitive impairment, particularly in patients diagnosed with schizophrenia. The invention is exemplified by the use of DAS-431 (or (−)-trans 9,10-diacetyloxy-2-propyl-4,5,5a,6,7,11b-hexahydro-3-thia-5-azacyclopent-1-ena[c] phenanthrene hydrochloride) which is a diacetyl prodrug of A-86929 (or (−)-trans 9,10-dihydroxy-2-propyl-4,5,5a,6,7,11b-hexahydro-3-thia-5-azacyclopent-1-ena[c]phenanthrene hydrochloride), a selective agonist for dopamine D1-like receptors.

### BACKGROUND

Cognitive impairment has been associated with dopamine dysregulation in several psychiatric diseases including schizophrenia, drug addiction, Parkinson's disease, depression, Huntington disease, as well as in normal aging (Goldman-Rakic et al. (2000) Brain Res Brain Rev 31:295-301). Dopamine is a chemical messenger generated during neurotransmission in the central nervous system (CNS) but also is involved in peripheral and vascular neurotransmission. In the dopaminergic system, dopamine passes by neurotransmission from a neuronal ending to the synaptic cleft and then to post-synaptic receptors located nearby on other neurons or on an excitable cell such as a muscle cell. Dopamine receptors are found at high levels in dopamine-rich regions of the brain and other areas of the CNS as well as in the peripheral nervous system and the renal and mesenteric vascular beds where large numbers of these receptors are found. The dopamine D1 receptor is located on the post-synaptic membrane; it is a transmembrane receptor comprised of seven peptide units which bind the G protein and stimulate adenylate cyclase and the production of cAMP when activated. The D1-like family of receptors includes two different receptors, named as D1 and D5 in humans and D1A and D1B in rodents. A variety of studies in animal models, as well as neuropsychological studies in clinical subjects, suggest a direct association between altered dopamine transmission in the prefrontal cortex and cognitive deficits in these diseases.

Working memory is the ability to hold an item of information transiently in mind in the service of comprehension, thinking, and planning. It encompasses both storage and processing functions, therefore subtle deficiencies in working memory can lead to substantial cognitive deficits in ideation, reasoning and planning. In humans and in primates, the frontal cortex is essential for working memory. More particularly, the prefrontal cortex mediates the cognitive processes of working memory (Goldman-Rakic, P. S. (1996) Proc Natl Acad Sci USA 93: 13473-13480). Several elements have suggested that dopamine may be the key neurotransmitter influencing the cognition processes. First, the density of dopamine D1 receptors is 10 to 20 times higher than that of dopamine D2 receptors in the cortex, including the prefrontal cortex (Lidow M S et al. (1991) Neuroscience 40:657-671). Second, local injections of selective dopamine D1 receptor antagonists (SCH23390 and SCH39166) into the prefrontal cortex of rhesus monkeys induce errors and increase latency of performance on working memory tasks (Sawaguchi and Goldman-Rakic (1991) Science 251:947-950, Sawaguchi (2001) Neurosci Res 41: 115-28). Third, the full D1 agonist dihydrexidine improves memory performance in young control monkeys and aged monkeys and these agonist-induced improvements are blocked by SCH23390, a specific dopamine D1 receptor antagonist (Arnsten et al. (1994) Psychopharmacology 116:143-151). In humans, a placebo-controlled, cross-over study on 32 healthy volunteers showed that 0.1 mg of pergolide, a D1/D2 R agonist, but not 2.5 mg of bromocriptine, a dopamine D2 receptor agonist, has a positive effect on spatial memory (Muller et al. (1998) J Neurosci 18:2720-2728). These finding suggest a preferential role for prefrontal dopamine D1 receptors for working memory modulation in humans.

In diseases such as schizophrenia, cognitive impairment, including working memory impairment, as measured by a variety of cognitive task performance tests (Condray et al. (1996) Schizophr Res 20:1-13; Weickert et al. (2000) Arch Gen Psychiatry 57:907-913) has been attributed to impairment of dopaminergic function in the prefrontal cortex (PFC). Moreover, these neurocognitive deficits have become increasingly important defining features of schizophrenia and its treatment. Schizophrenia is a chronic disease that is characterized by positive (hallucinations, delusions), negative (social withdrawal, flattened affect) and cognitive (formal thought disorder, executive memory dysfunction) symptoms. Multiple domains of cognitive function are impaired in schizophrenia and these impairments are considered to be core features of the disorder. Certain neurocognitive domains such as verbal working memory, immediate memory, executive functioning and vigilance are associated with functional outcomes. Indeed, cognitive dysfunction, rather than clinical symptoms, may be the most important factor in the ability of a patient with the disease to be able to perform their daily tasks.

Antipsychotic drugs are often effective in treating certain symptoms of schizophrenia, particularly hallucinations and delusions; unfortunately, the drugs may not be as helpful with other symptoms, such as reduced motivation and emotional expressiveness. Indeed, the older antipsychotics (which also went by the name of “neuroleptics”), including haloperidol (Haldol®) or chlorpromazine (Thorazine®), may even produce side effects that resemble the more difficult to treat symptoms. A number of new antipsychotic drugs (the so-called “atypical antipsychotics”) have been introduced since 1990. The first of these, clozapine (Clozaril®), has been shown to be more effective than other antipsychotics, although the possibility of severe side effects—in particular, a condition called agranulocytosis (loss of the white blood cells that fight infection)—requires that patients be monitored with blood tests every one or two weeks. Even newer antipsychotic drugs, including olanzapine (Zyprexa®), quetiapine (Seroquel®), and risperidone (Risperdal®), are safer than the older drugs or clozapine, and they also may be better tolerated. It is unclear whether they treat the illness as well as clozapine, however.

The drugs that are currently used for treating schizophrenia have various effects on cognitive function. Typical antipsycotic neuroleptic drugs lack any ability to improve the domains of cognitive function that are impaired in schizophrenia. Atypical antipsychotic drugs, on the other hand, do exhibit some, albeit heterogeneous, positive side effects on cognitive function. Clozapine improves attention and verbal fluency, but tests of its effect on working memory have been inconclusive. Risperidone showed positive effects on working memory and executive functioning, but inconsistent results were obtained in tests of verbal learning and memory. Olanzepine improves verbal learning and memory, and executive function, but not the working memory. One possible explanation for these effects may be an indirect and moderate D1 receptor agonist property exhibited by some of the atypical antipsychotic agents. Although an incidental agonistic effect on dopamine receptors might partially explain the effectiveness of atypical antipsychotic drugs, none of these drugs was specifically designed to improve cognitive function. Rather, one of the goals of treatment with antipsychotic drugs is to prevent hallucinations and delusions. Addressing these symptoms, however, is not the only, or necessarily the most appropriate, approach to treating schizophrenia; while drugs that blunt the mind may effectively diminish some symptoms of schizophrenia, drugs that also enable clear thinking would be preferable.

Use of a drug that directly targets cognitive function deficiencies offers a different approach to current treatments for treatment of schizophrenia and other diseases or states that present with a symptom of cognitive impairment. Such a drug is DAS-431 and related selective D1 receptor agonists. DAS-431 has been used to treat several disorders relating to dopaminergic status, including Parkinson's disease and cocaine abuse. However, with the treatment protocols used, as a potent dopaminergic agonist, DAS-431 has been associated with a high incidence of dopamine related symptoms such as nausea, vomiting, hypotension, postural hypotension, dizziness, injection and application site reactions. Transitory EKG changes also have been observed. These adverse events were commonly described as mild or moderate. Though mild and moderate in nature, adverse events are of concern for use in target diseases such as schizophrenia, and even more so for use in pediatric populations and treatment of cognitive impairment in the normal aging population. It therefore is of interest to develop treatment regimes that act by directly improving cognitive function while minimizing the side effects of treatment and in the case of schizophrenic patients while additionally still controlling symptoms including hallucinations and delusions.

### Relevant Literature

The use of D1 agonists for the treatment of schizophrenia has been proposed, but there are no reports on the efficacy of treatment with D1 agonists nor reports of the utility of D1 agonists in the treatment of memory and cognition in humans. Improved memory performance in monkeys following administration of D1 agonists such as dihydrexidine and the partial agonist SKF 38393 have been reported. The group of Patricia Goldman-Rakic tested DAS-431 in a non-human primate model of cognitive impairment (Castner et al. (2000) Science 287:2020-2022.). In this study, severe impairments in working memory were induced by chronic treatment with haloperidol. DAS-431 (0.01 μg/kg to 0.1 μg/kg IM) was reported to reverse the haloperidol effects. The treatment regimen used five blocks of five consecutive days of treatment with a minimum wash out period of 2 weeks between blocks. The effect of treatment with DAS-431 persisted in some monkeys for more than a year after the final treatment. Conversely, in one experiment, assessing DAS-431's efficacy as a treatment for ethanol reinforced behavior in monkeys, the data presented suggested that tolerance may have developed after 3 days of treatment (0.1, 0.3 and 1 mg/kg/day IM). Other studies using DAS-431 show that the potent effect is maintained following repeated administration (Michaelides et al. (1995) J Med Chem 38:3445-3447). Particularly, an experiment on MPTP-lesioned macaques showed no difference over time on DAS-431 efficacy after a three-time daily administration (0.32 mg/kg /s.c) for thirty days.

U.S. Pat. Nos. 5,668,141 and 5,597,832 disclose that 2,6-, 3,6- and 4,6-diaza-5,6,6a,7,8,12b-hexahydrobenzo-[c]phenanthrene compounds and DAS-431 and related compounds are useful agents for the treatment of dopamine-related neurological, psychological and cardiovascular disorders. There is no disclosure relating specifically to treatment of cognitive disorders, including cognitive impairment in schizophrenia, or of treatment regimens to minimize side effects and/or provide long-term efficacy.

An intravenous formulation of DAS-431 was tested in humans with doses ranging from 1 mg to 40 mg IV per day, in three different populations: normal volunteers (max. dose 5 mg), Parkinson's disease subjects (max. dose 40 mg) and cocaine users (max. dose 32 mg). DAS-431 was administered as a single daily one-hour IV infusion. In phase II trials, the compound was tested in two subject populations: patients with Parkinson's disease (Rascol et al. (1999) Ann Neurol 45:736-741; Rascol et al. (2001) Arch Neurol 58:249-254) and chronic cocaine users (Haney et al. (1999)Psychopharmacology (Berl) 143:102-110; Malison et al. “Reductions in Cocaine-induced craving following the selective D1 Dopamine receptor agonist ABT-431 in Human cocaine abusers”). The maximal effective IV dose of DAS-431 on motor activity in subjects with Parkinson's disease was 30 to 40 mg per day. In the cocaine user population, the potentially effective IV dose, which reduced cocaine craving and cocaine intent to use, was 4 to 8 mg per day. Though there is a higher incidence of adverse events at higher doses, there is no clear dose relationship with the exception of nausea and vomiting in the cocaine abuser population and of dizziness in all subjects. Moreover, the studies seem to show a rapid tolerance to nausea and vomiting in the Parkinson's disease population. The incidence of side effects is greater in the Parkinson's disease population, due mainly to the fact that the disease itself is associated with hypotension and postural hypotension, and to the fact that study subjects took DAS-431 in addition to their chronic anti-Parkinson's medications.

Dopamine D1 receptor partial agonists such as SKF 38393 (optionally in conjunction with haloperidol, a D2 antagonist) have been used in the treatment of schizophrenia and have been reported to decrease food intake in rats. However, SKF 38393 was without anti-parkinsonian activity in an MPTP treated monkey model for Parkinson's Disease. MPTP treated monkeys have unequivocal signs of parkinsonism, including tremor, poverty of movement, difficulty in initiating movement, bradykinesia, motor freezing and a decrease in eye blink rate. Dihydrexidine attenuates these parkinsonian signs while increasing eye blink rate. These effects were blocked by the dopamine D1 receptor antagonist SCH 23390 but not by the dopamine D2 receptor antagonist remoxipride. SKF 82958, a high efficacy partial D1 agonist, has been reported to increase locomotor activity and improve parkinsonian score following administration in MPTP-treated primates. D1 agonists, including DAS-431, have been reported to have antiparkinsonian activity.

Dopamine D1 receptor antagonists also have been used in the treatment of CNS disorders. As an example, the dopamine D1 receptor antagonist SCH 23390 had profound behavioral effects in schizophrenic patients, but other dopamine D1 receptor antagonists were without effect.

### SUMMARY OF THE INVENTION

Methods are provided for the long term treatment of cognitive impairment in a patient in need thereof using selective dopamine D1-like receptor agonists and/or prodrugs thereof, a profile and methods for developing a profile that correlates dopaminergic state to cognitive impairment and methods for predicting response to provision of selective dopamine D1-like receptor agonists by determining catechol-o-methyltransferase (COMT) genotype of the patient. The method of treatment includes the step of providing to a patient in need thereof an amount of one or more selective dopamine D1-like receptor agonist effective for treating the cognitive impairment using a dosing regimen that minimizes side effects and/or desensitization or tolerance to the treatment and/or provides for long term improvement in cognitive function and/or prevention of further deterioration of cognitive function. Preferably other symptoms of the underlying disease that is associated with the cognitive impairment also are improved. Alternatively, two or more drugs with different mechanisms of action can be combined in the same treatment, where one of the drugs is a selective dopamine D1-like receptor agonist effective for treating the cognitive impairment and the second is a drug with some efficacy for treating the underlying disease state. In combination these drugs can act additively or synergistically. The method of generating a profile includes the steps of determining the dopaminergic status and/or the cognitive function of both individual patients with cognitive impairment and of a population of patients with cognitive impairment associated with a particular disease state or normal aging and correlating that status cognitive function both in the individual patient and in the particular population of patients. The methods find use in the treatment of cognitive impairment in several diseases including schizophrenia, substance addiction and abuse, Parkinson's disease, depression, Huntington disease, as well as in normal aging in patients who have clear cognitive impairments of at least one of working memory, verbal and spatial memory and executive functioning. The profile can be used in patient diagnosis, treatment and prognosis and in identifying optimal treatment regimens for individual patients with cognitive impairment and for particular diseases where a presenting symptom is cognitive impairment.

### DESCRIPTION OF THE PREFERRED EMBODIMENTS

Methods are provided for alleviating and/or slowing progression of at least one symptom, including cognitive impairment, of schizophrenia and other diseases such as depression and/or drug and/or alcohol abuse and/or addiction in which a presenting symptom is cognitive impairment using selective dopamine D1-like receptor agonists and/or prodrugs thereof either alone or in combination with other treatment modalities. By cognitive impairment is intended inability to retain and manipulate information over a brief period of time as measured by a test of working memory such as the n-back test. By a schizophrenic patient is intended a patient diagnosed with schizophrenia or shizophreniform disorder according to the Diagnostic and Statistical Manual (DSM-IV). Generally the cognitive impairment is associated with decreased density of dopamine D1 receptors in areas of the brain associated with working memory, including the frontal cortex, and particularly the prefrontal cortex (PFC). The methods of treatment include the use of DAS-431, a dopamine D1 receptor agonist chemically related to DAS-431, or an other dopamine D1 receptor agonist, either alone or in combination with a second drug, typically a drug that shows some efficacy in treating symptoms of the presenting condition, but not necessarily cognitive impairment.

In the methods, a schizophrenic patient, or a patient with cognitive impairment due to other causes such as Alzheimer's disease, amyotrophic lateral sclerosis, a brain injury, a degenerative disorder associated with a learning and memory deficit, autism, age-related cognitive decline, mild cognitive impairment, alcohol and/or drug addiction or abuse, electric shock induced amnesia, head trauma, stroke, cerebral ischemia, Huntington's disease, a learning disability, multi-infarct dementia, senile dementia, Parkinson's disease, Tourrett's syndrome, or major depression is provided with a formulation which includes at least one selective dopamine D1-like receptor agonist and/or prodrug thereof in a dose which is in a range and using a treatment regimen which is appropriate for the PFC dopaminergic status of the patient. Optionally the patient is also provided with other compositions suitable for treatment of the patient's disease or state, generally compositions with a mechanism of action other than that of a selective dopamine D1-like receptor agonist. By PFC dopaminergic status is intended a profile relating PFC dopamine D1 receptor density in an individual patient and the degree of cognitive impairment (preferably working memory/executive functioning) relative to the dopaminergic status for a population of patients with the same disease or cause of decreased cognitive function. Population profiles of dopaminergic status associated with disease severity or other disease specific characteristics thus can be developed. The specific disease characteristics included depend on the disease.

The response of individuals within the population to various treatment protocols is an important factor in profiling the relationship between dopaminergic status of an individual and their responsiveness to treatment. Individuals who respond poorly to treatment, for example, may have a dopaminergic status that makes them poorer candidates for a particular treatment regimen than patients with a dopaminergic status similar to that of individuals who respond well to that same treatment regimen. Generally, population studies are required to establish these relationships between dopaminergic status and response to treatment with a reasonable degree of significance. Once a relationship between dopaminergic status and treatment response is established in a population, the selection of a treatment for any given patient can be improved by determining the dopaminergic status for an individual patient using appropriate cognitive tasks. Alternatively, dopaminergic status can be determined by determining the dopamine D1 receptor density in an individual patient, for example using Positron Emission Tomography (PET) while the working memory of the patient is assessed. Those treatment regimens that have been established as successful for individuals with substantially similar profiles to that of the patient are most likely to prove efficacious.

The subject invention has several advantages over existing methods for treating schizophrenia and other diseases in which a presenting symptom is cognitive impairment. In the subject methods, cognitive function in, for example schizophrenia, is improved as opposed to blunted as is typically the case with treatment with current antipsychotic drugs, while other symptoms such as hallucinations and delusions are still controlled. The enhancement of cognitive function benefits patients by improving their ability to perform necessary tasks.

Additional advantages include that the dosages that are used are significantly lower than those that are traditionally used and thus there are less side effects and less chance of a patient developing tolerance to the treatment and the concomitant need to increase the dose, and hence increase the likelihood of side effects, to maintain efficacy of the treatment. With less side effects, patient compliance increases, leading to better control of symptoms. The treatment regimen employed additionally has the advantage that chronic treatment is not required; the effects of the regimen are long lasting. The use of less drug per dose and is of a decreased number of doses offer the additional advantage of decreased cost of therapy.

The development of profiles of diseases that affect working memory and correlate the dopaminergic status and the degree of cognitive impairment offers additional advantages. One such advantage is the feasibility of more precise treatment protocols for patients with impaired cognitive function related to that disease. Another advantage is the potential for treatment of individual patients within the population whose individual profile differs significantly from the norm for that population. Optionally using the identification of a COMT genotype status that predisposes an individual patient to difficulty in performance of working memory tasks, offers the advantage that patients can be identified who are in need of an alternate treatment and/or dosing regimen from those traditionally prescribed.

Pharmaceutical compositions of the invention are suitable for use in a variety of drug delivery systems. Pharmaceutically acceptable carriers and formulations that find use in the present invention are found in Remington's Pharmaceutical Sciences, Mack Publishing Company, Philadelphia, Pa., 17th ed. (1985), which is incorporated herein by reference. For a brief review of methods for drug delivery, see Langer, (1990) Science 249:1527-1533, which is incorporated herein by reference.

In preparing pharmaceutical compositions of the present invention, it may be desirable to modify the compositions of the present invention to alter their pharmacokinetics and biodistribution. For a general discussion of pharmacokinetics, see Remington's Pharmaceutical Sciences, supra, Chapters 37-39. A number of methods for altering pharmacokinetics and biodistribution are known to one of ordinary skill in the art (See, e.g., Langer, supra). Examples of such methods include protection of the agents in vesicles composed of substances such as proteins, lipids (for example, liposomes), carbohydrates, or synthetic polymers. For example, the agents of the present invention can be incorporated into liposomes in order to enhance their pharmacokinetics and biodistribution characteristics. A variety of methods are available for preparing liposomes, as described in, e.g., Szoka et al, (1980) Ann. Rev. Biophys. Bioeng. 9:467, U.S. Pat. Nos. 4,235,871, 4,501,728 and 4,837,028, all of which are incorporated herein by reference.

The agents of the present invention can be used in pharmaceutical compositions that are useful for administration to humans. See U.S. Pat. No. 5,597,832. Compounds of the present invention can be administered to a mammalian host in a variety of forms adapted to the chosen route of administration, e.g. orally or parenterally. Parenteral administration in this respect includes administration by the following routes: intravenous, intramuscular, subcutaneous, intraocular, intrasynovial, transepithelial including transdermal, ophthalmic, sublingual and buccal; topically including dermal, rectal and nasal inhalation via insufflation, aerosol and rectal systemic.

The invention provides compositions that comprise a solution of the agents described above dissolved or suspended in an acceptable carrier, preferably an aqueous carrier. A variety of pharmaceutically acceptable aqueous carriers can be used, e.g., water, buffered water, 0.4% saline, 0.3% glycine, hyaluronic acid and the like. These compositions can be sterilized by conventional, well-known sterilization techniques, or can be sterile filtered. The resulting aqueous solutions can be packaged for use as is, or lyophilized, the lyophilized preparation being combined with a sterile solution prior to administration. The compositions can contain as pharmaceutically acceptable carriers, substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like.

For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient and more preferably at a concentration of 25%-75%.

The compounds of this invention can be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as a membrane. The active agent also can be provided as a transdermal patch that can include the compound in a suitable solvent system with an adhesive system, such as an acrylic emulsion, and a polyester patch.

The compounds of the present invention also can be delivered through mucosal membranes. Transmucosal (i.e., sublingual, buccal, and vaginal) drug delivery provides for an efficient entry of active substances to the systemic circulation and reduces immediate metabolism by the liver and intestinal wall flora. Transmucosal drug dosage forms (e.g., tablet, suppository, ointment, pessary, membrane, and powder) are typically held in contact with the mucosal membrane where they disintegrate and/or dissolve rapidly to allow immediate systemic absorption. For delivery to the buccal or sublingual membranes, typically an oral formulation, such as a lozenge, tablet, or capsule, is used. The method of manufacture of these formulations is known in the art, including the addition of the pharmacological agent to a pre-manufactured tablet, cold compression of an inert filler, a binder, and either a pharmacological agent or a substance containing the agent (see for example U.S. Pat. No. 4,806,356); and encapsulation. Another oral formulation is one that can be applied with an adhesive, such as the cellulose derivative hydroxypropyl cellulose, to the oral mucosa (see for example U.S. Pat. No. 4,940,587). This buccal adhesive formulation, when applied to the buccal mucosa, allows for controlled release of the pharmacological agent into the mouth and through the buccal mucosa.

For aerosol administration, the pharmaceutical compositions are preferably supplied in finely divided form together with a surfactant and propellant as pharmaceutically acceptable carriers. The surfactant is nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides, can be employed. A carrier also can be included, as desired, as with, for example, lecithin for intranasal delivery. An especially preferred polysaccharide excipient is cross-linked starch microspheres, such as those described in GB-1518121. The microspheres in GB-1518121 are produced by the emulsion polymerization of a soluble potato starch hydrolysate to give microspheres which are then cross-linked, e.g. by means of epichlorohydrin. Further details can be found in Lindbergh et al., Microspheres and Drug Therapy, Davis et al., Elsevier, Amsterdam, 1984, p 153. See also U.S. Pat. No. 6,310,089 which discloses an intranasal formulation of dopamine D1 receptor agonists such as DAS-431 (referred to as ABT-431).

To treat a patient in need thereof, the pharmaceutical compositions of the subject invention can be administered in a variety of unit dosage forms depending upon the method of administration. The effective amount of a particular agent in a pharmaceutical composition depends on, for example, the chemical nature of the agent, the manner of administration, the weight and general state of health of the patient, the severity of the cognitive impairment being treated and the judgment of the prescribing physician. Dosages, formulations and administration schedules can vary in particular patients as compared to normal individuals and/or other patients.

For treatment with DAS-431, generally intravenous administration or preferably dermal or transmucosal delivery is used. The dosages for intravenous administration range from about 1 μg to about 50 mg or more, preferably 5 μg to about 20 mg with dosages of from about 30 μg to about 8 mg (i.e. about 0.00001 μg to 0.10 mg/kg of body weight) being more commonly used. However, the compositions of the present invention may be employed in serious disease states, and in such cases it is possible and may be felt desirable by the treating physician to administer substantial excesses of these compositions over the dosages recited above. Depending upon the dose that is used, IV administration is usually given over about 0.5 to 2 hours, preferably over about an hour to minimize side effects, such as nausea and local injection site reactions. If a different route of administration is used, such as dermal or transmucosal, the dosages are adjusted accordingly to compensate for efficiency of administration, and greater or lesser bioavailability by the chosen route as compared to intravenous administration. If neuroimaging is to be used to evaluate treatment efficacy, optionally a patient receives an antinausea medication such as odansetron prior to neuroimaging to prevent potential side effects, which could prevent acquisition of high quality images.

In addition to treatment with a selective dopamine Dl receptor agonist such as DAS-431 or similar compounds, a patient optionally can be treated with a second drug known to be efficacious for the disease or state being treated, particularly drugs that have a different mechanism of action than does DAS-431. As an example, when the underlying disease is schizophrenia, the second drug may be a selective dopamine D2 receptor antagonist such as haloperidol or risperidone, or an atypical antipsychotic, such as olanzapine or ziprasidone can be administered. For treatment of cognitive impairment in diseases or states other than schizophrenia, DAS 431 or another selective D1 receptor agonist can be combined with approproiate medication for the disease or state such as neuroleptics, lithium, antiepileptics, benzodiazepine, hypnotics, antidepressants, etc. The two (or more) drugs (selective D1 receptor agonist and another drug) can be administered in one composition or as two separate entities. For example, they can be administered together in a single infusion or as individual compounds. The components included in a particular composition, in addition to the selective dopamine D1 receptor agonist and another drug, are determined primarily by the manner in which the composition is to be administered as described above.

The treatment regimen that is used for treating cognitive impairment takes into account that stimulation of dopamine receptors in the PFC produces an inverted “U” shape dose response curve: either insufficient or excessive dopamine D1 receptor stimulation is detrimental to PFC cognitive function; a narrow range of concentrations is available for optimal dopamine D1 receptor stimulation. The dose response curve for individual patients is also dependent on the patient's PFC dopaminergic state. Thus the dose response curve for treating each patient is preferably determined empirically by combining information concerning the generally efficacious range with information regarding the patient's PFC dopaminergic state. An effective dose for most patients is less than about one mg per day. The therapeutic range is determined by measurements of the effect of different drug concentrations on symptoms of patients. Individual adjustments are then performed to reflect the responsiveness of particular patients to treatment. Typically, the initial treatment that is used generally is toward the low end of the dosage scale that is efficacious for the particular cognitive impairment. If no or minimal improvement is seen with the initial treatment, it can be repeated with the same dose at about 12-36 hour intervals, generally about 24 hour intervals and the evaluation repeated. If there is no or a minimal effect from treatment, the dose can be increased after one to two weeks and this process is repeated until there is clear improvement and/or normalization on the working memory task. If by increasing the dose a deleterious effect is observed, the dose should be decreased to the previous dose at which efficacy was observed. If the initial dose was not effective, and increasing the dose was deleterious, then the dose can be decreased below the initial dose until an efficacious dose is reached. In some patients, only deleterious effects may be observed, and treatment in such patients should be discontinued and treatment with an alternate medication such as a dopamine D1 receptor antagonist evaluated instead.

In some patients, tolerance to repeat treatment may be noted. A single daily administration would limit the occurrence of tolerance. In this instance, treatment can be halted for a time sufficient for the treatment drug to be eliminated, generally about five days to ten days. Treatment can be repeated, whether daily or with wash out periods, until there is stable improvement in cognitive function for at least a week at which time treatment can be halted. Following treatment, the patient is monitored for maintenance of the improved cognitive function at regular intervals, generally about every 1 to 3 months. If a decline in cognitive function is noted, the course of treatment can be repeated, generally with the same dosage found to be efficacious previously for this patient.

The effect of treatment of symptoms of impairment of working memory can be evaluated as follows. Both the biological efficacy of the treatment modality as well as the clinical efficacy are evaluated, if possible. For example, dopamine D1 receptor stimulation is associated with improvement of working memory as measured by n-back performance. Moreover, it is a theory of the invention that there is a relationship between occupancy of dopamine D1 receptors and working memory improvement. To most accurately measure the effect of treatment, in general, prior to treatment, the cognitive function or dopaminergic status of a patient is determined by any of a number of techniques for measuring working memory and/or executive functioning impairment. The same tests performed prior to treatment are repeated following treatment to evaluate if there is an improvement in cognitive function and/or dopaminergic status. Optionally, the COMT genotype of patients may be determined to select patients most likely to benefit from treatment (see Examples). Patients determined to be homozygous with the val allele are likely to respond differently to treatment as compared to heterozygous or met allele homozygous patients (see for example Egan M F et al. (2001) Proc Nat Acad Sci USA 98:6917-6922; Gogos J A et al. (1998) Proc Natl Acad Sci USA 95:9991-9996; Kneavel et al. (2000) Society for Neuroscience 30^{th} Annual Meeting; 571.20).

Examples of tests for measuring cognition include the following: Brief Psychiatric Rating Scale, Clinical Global Impression, Positive and Negative Symptoms Scale, Scale for Assessing Negative Symptoms, Young Mania Rating Scale, Cognitive subscale of the Alzheimer's Disease Assessment Scale, Clinician's Interview Based Impression of Change, Short Portable Mental Status Questionnaire, Folstein Mini-Mental Status Examination, Clinical Dementia Rating scale, Cambridge Neuropsychological Test Automated Battery, Wisconsin Card Sort Test, N-back working memory test, Weather prediction probabilistic learning test, Repeatable Battery for Assessment of Neuropsychological Status, or Continuous Performance Test Vigillance. As the range of cognitive impairment domains may vary from one disease to another, additional cognitive tasks may be added to any test for a complete treatment of multiple cognitive impaired domains. Optionally, brain activity can also be evaluated concurrently using a neuroimaging tool such as functional magnetic resonance imaging (fMRI), or a PET scan using a radiopharmaceutical such as or F-18N-methylspiperone so as to provide the dopaminergic status of the patient. It is a theory of the invention that a patient in whom an increase in the number of dopamine D1 receptors in the PFC (i.e. receptor upregulation) is observed following treatment is most likely to show an improvement in working memory performance and that the dopaminergic status in any particular disease state is correlated with the degree of cognitive impairment.

The clinical efficacy, whether treatment of the underlying defect is effective in changing the course of disease, can be more difficult to measure. While the evaluation of the biological efficacy goes a long way as a surrogate endpoint for clinical efficacy, it is not definitive. Thus, measuring a clinical endpoint which can give an indication of improvement in working memory after, for example, a six-month period of time, can give an indication of the clinical efficacy of the treatment regimen. The extent and progression or regression of symptoms of working memory impairment are evaluated and monitored by cognitive tests, such as those mentioned above. In terms of clinical features of schizophrenia, the positive and negative symptoms can be measured by a series of scales such as GCI (Global Clinical Impression) PANSS (positive and negative syndrome Scale) plus several subsets for negative symptoms as measured by, for example, BPRS (Brief psychiatry rating Scale). With the derived versions for the total score and for the negative symptoms, absence of progression or regression of these changes after a period in which they generally would be expected to develop in a patient with a particular level of schizophrenia can give an indication of the clinical efficacy of the treatment regimen. Similarly, one skilled in the art can evaluate the biological and clinical efficacy of a treatment for other diseases with a presenting symptom of cognitive impairment.

The subject compositions can be provided for use in one or more procedures. For treatment with a pharmaceutical composition comprising an agent identified as one which is effective in directly or improving the symptoms of cognitive impairment, the subject compositions can be provided as kits for use in one or more doses. The kits include a composition comprising an effective agent either as concentrates (including lyophilized compositions), which may be further diluted prior to use or they may be provided at the concentration of use, where the vials may include one or more dosages. Conveniently, in the kits single dosages can be provided in sterile vials so that the physician may employ the vials directly, where the vials will have the desired amount and concentration of agents. When the vials contain the formulation for direct use, usually there will be no need for other reagents for use with the method. The kits also can be in the form of a transdermal or transmucosal system for single or multiple applications. The subject compositions can be contained in packaging material, which comprises a label indicating that the subject compositions can be used to treat cognitive disorders in humans.

The following examples are offered by way of illustration and not by way of limitation.

### EXAMPLES

### Example 1

### Measurement of DAS-431 Receptor Occupancy In Vivo

The purpose of this study is to measure the in vivo occupancy by potential therapeutic doses of an intravenous (IV) formulation of DAS-431 (4 and 8 mg) of the D1 receptor using PET imaging and the D1 specific radiotracer [11C]NNC 112. This is a Phase IIa, open label study, each subject being their own control, in different subject population. From the data obtained, potential therapeutic doses can be calculated and extrapolated to putative effective doses in non-schizophrenic populations. A total of 20 treated subjects (max. total 28 recruited) in four subject populations are studied:

Group 1: Schizophrenic subjects (5 treated)

Group 2: Cocaine dependent subjects (5 treated)

Group 3: Matched healthy volunteers (controls) (5 treated)

Group 4: Aged healthy volunteers (5 treated)

Subjects are eligible to enroll in the study if they are male or female between 18 and 65 years old. For Schizophrenic subjects, they must meet DSM-IV criteria for schizophrenic illness, schizophreniform or schizoaffective disorder and have negative urine toxicology. For cocaine dependent subjects, they are eligible to enroll if they fulfill the DSM-IV criteria for cocaine dependence. For healthy volunteers, they are eligible if they have negative urine toxicology. For all groups, the subjects must be free of antipsychotic medications in the last 3 weeks or of depot neuroleptics in the last 6 months, be free of antidepressants or mood stabilizers in the last 4 weeks, of fluoxetine in the last 6 weeks, have no history or presence of seizures, cancer or any clinically significant cardiac, respiratory, metabolic, renal, hepatic, gastrointestinal, dermatological, venereal, hematological, neurological or psychiatric disease or disorder (other than as specifically mentioned above), not have a diastolic blood pressure ≧90 mm Hg and/or a systolic blood pressure ≧140 mm Hg or a decrease of 30 mm Hg or greater in systolic blood pressure after arising from a 5-minute supine position to a 1-minute standing position, have no metal or paramagnetic objects within the body that would interfere with the MRI scan, and have no current, past or anticipated exposure to radiation in the workplace, or participation in nuclear medicine procedures.

DAS-431 is provided as a sterile lyophilized preparation in 10 mL single use vials, containing 6.9 mg DAS-431 (the equivalent of 5 mg A-86929) and 100 mg mannitol. DAS-431 is mixed in sterile water for injection usp and administered as a 60 minute IV infusion. It is administered in doses of 4 or 8 mg of DAS-431 as described below for each group. All subjects receive 8 mg po odansetron prior to each PET scan, to prevent DAS-431 potential side effects (nausea, vomiting), which could prevent acquisition of high quality images.

Schizophrenic subjects and matched controls (groups 1 and 3) are treated for 2 consecutive days, receiving a single administration of 4 mg the first day and one of 8 mg the second day of DAS-431. D₁ receptor measurements using PET imaging is carried out essentially as described by Abi-Dargham et al (above) aa: 3708-3719, using (+)-5-(7-Benzofuranyl)-8-chloro-7-hydroxy-3methyl-2,3,4,5-tetrahydro-1H-3-benzazephine (NNC 112), a potent and selective D₁ receptor antagonist (Andersen et al. (1992) Eur J Pharmacol 219:45-52). [¹¹C]NNC 112 is used as a radio tracer to image D₁ receptors (Halldin et al. (1998) J Nucl Med 39:2061-2068). [11C]NNC 112 is administered as an IV bolus at a maximal dose of 6.54 μg. On each of two separate days, these subjects undergo 2 PET scans on the HR+ camera, following injection of 20 mCi [11C]NNC112 to examine regional D1 receptor binding potential at baseline, and then after administration of a low dose of DAS-431 (4 mg) on the first day, or a higher dose of DAS-431 on the second day (8 mg). Cocaine dependent subjects and healthy volunteers (groups 2 and 4) are treated for a single day, with 4 mg of DAS-431. [11C]NNC 112 is administered as an IV bolus at a maximal dose of 6.54 μg and the subjects undergo 2 PET scans on the HR+ camera, following injection of 20 mCi [11C]NNC112 to examine regional D1 receptor binding potential at baseline, and then after administration of DAS-431 (4 mg). No comparative therapy is evaluated.

The efficacy of treatment is evaluated by the dopamine D1 receptor binding potential. The safety of the treatment is measured by following vital signs, and performing full chemistry and hematological testing, EKG, and urine analysis. Pharmacokinetics are determined by taking samples during the infusion and PET Scan acquisition at the indicated times: 0, 30, 45, 55, 60, 65, 75, 90, 120 min after radiotracer injection, for determining A-86929 plasma levels.

The data are analysed as follows. Occupancy of DAS-431 at prefrontal D1 receptors is calculated as [100*(BPbaseline−BPDAS−431)/BPbaseline]. Quantification of [11C]NNC 112 BP is performed as described (Abi-Dargham et al. J Cereb Blood Flow Metab 2000; 20:225-43). Individual analysis: For each subject in group 1 and group 3, significant differences in Emax and ED50 are tested between subjects with schizophrenia and normal volunteers. Group analysis: Data is by diagnosis group, under the assumption of no significant difference within groups in Emax and ED50. Group level Emax and ED50 are derived, and between group differences are estimated.

### Example 2

### DAS-431 Action on Cognitive Performance and on Normalizing Abnormal rCBE Patterns in Frrontal Cortex in Risperidone Treated Schizophrenic Subjects

The purpose of this study is to evaluate the effect of (1) a single rising dose of DAS-431 and (2) a five day treatment with DAS-431 on symptoms of schizophrenia, on cognitive performance and on CNS physiology, by region and by dose, both at rest and during performance of auditory and memory tasks. This is a phase IIa, two-step study.

The initial phase is within subject randomized placebo controlled assessment of the effect of a single rising daily dose of DAS-431: 2, 4, 8 mg. Each testing day is 2 or 3 days apart. The second phase starts after a week wash-out and is a double blind, randomized, parallel, placebo controlled study. All subjects receive 5 consecutive days of placebo followed by five consecutive days of DAS-431 treatment, at one of the three tested doses or a placebo. A total 20 subjects is tested, 5 per group with 3 active groups and 1 placebo group. As required, subjects are replaced to obtain final sample size.

Subjects are eligible to enroll in the study if they: are in-patient, risperidone treated schizophrenic subjects; meet DSM IV schizophrenia criteria with active psychotic symptoms, assessed by complete set of clinical symptoms rating, BPRS, SANS and psychosis change scale; show attentional deficit on the CPT and working memory impairment on the Hopkins verbal learning task. Patients with predominantly negative symptoms are excluded.

DAS-431 IV infusion, 2, 4 and 8 mg daily, is administered over 60 minutes. A PET scan is done at a plateau concentration over 90 minutes. All subjects receive odansetron prior to each PET scan, to prevent DAS-431 potential side effects (nausea, vomiting), which could prevent acquisition of high quality images. The comparative therapy is placebo, administered as the active treatment. The background treatment of the subjects is Risperidone (stable dose for at least 2 weeks).

Cognitive function, CPT and Hopkins verbal learning task are used to evaluate the treatment. Efficacy (time point evaluation) is determined using a 15OPET Scan under two sets of tasks: auditory tone, at three demanding levels and working memory tasks, N-back for word and string of letters and for clinical outcome using BPRS, SANS, and psychosis change scale. In the acute initial phase, all criteria are assessed of and on drug on the day of dosing.

In the subchronic phase, cognitive function and scan acquisition are performed on day 5 of each week (Placebo and DAS-431) and the clinical outcome is assessed on days 2, 4 and 5 of each week. To evaluate the safety of the treatments, vital signs, full chemistry and hematological testing, EKG, urine analysis are assessed at screening, each day of dosing at initial phase and on days 1 and 5 of each subchronic week Pharmacokinetic analysis is performed during and after the infusion, 0, 15, 30, 45, 60, 75, 90, 120, 135 and 150 minutes for A-86929 plasmatic levels. The primary endpoints are rCBF changes by region and dose, both at rest and during performance of task; cognitive testing for attention and working memory; and clinical outcomes.

### Example 3

### Acute and Subchronic Cyclical Pharmacological Modulation of Dopamine D1 Receptors

The purpose of this study is to evaluate acute and subchronic cyclical dopamine D1 receptor pharmacological modulation using low doses of DAS-431, as well as the effects on prefrontally mediated cognitive function, electrophysiological correlates and clinical outcomes in schizophrenic subjects. The objectives are as follows:

(1) To examine whether single dose administrations of DAS-431 have significant effect on cognitive and electrophysiological measures of prefrontal cortical function in healthy and schizophrenic subjects;

(2) To assess subject prefrontal dopaminergic state and to potentially use this as a predictor of cognitive, electrophysiological and clinical response.

(3) To examine whether a dosing regimen of very low dose and cyclical subchronic administration is effective and whether its effect is maintained after discontinuation of treatment.

This is a phase IIa, two-phase study. The initial phase is a double blind, within subject, placebo controlled study of single daily doses of DAS-431. The later phase is a randomized, parallel, double-blind, placebo controlled study of five courses of five consecutive days of treatment, nine days apart with three month post-treatment follow up. The patients will be institutionalized for the initial acute assessment and outpatient for the subchronic treatment.

The subjects for the study are 40 to 100 screened schizophrenic subjects.

(1) Acute phase: total 65 schizophrenic subjects—total 15 healthy volunteers (four treatment groups, plus one placebo, plus four treatment groups and one placebo of healthy volunteers).

(2) Main study: total 56 schizophrenic subjects (min. 45 completed) in three groups of 18/15 per group.

Subjects are eligible to enroll in the study if they meet DSM IV criteria for schizophrenia, are on stable long-term (at least 12 months) antipsychotic medication other than clozapine with impairment in prefrontally mediated cognitive function (greater than half standard deviation impairment), with a characterized baseline dopaminergic state.

Administration of the DAS-431 is by IV infusion over 30 minutes.

(1) Initial study: 0.005, 0.05 and 0.5, 1 mg, single daily administration—five consecutive days.

(2) Main study: 0.05 and 1 mg—five courses of five consecutive days, nine days apart. Placebo is administered as the test product.

The criteria for subject evaluation are (a) Working memory/executive function (CANTAB, other TBD); (b) ERP—working memory behavior: brain activity related to working memory/cotangent negative variation in a memory task with variable load, mismatched negativity; and (c) Clinical symptoms: GCI, SANS, PANNS, subset scores.

In the initial phase, all clinical criteria are assessed before after and after drug administration, each dosing day. In the main study, the cognitive function and ERP are assessed on day 1 (before treatment) and day 5 of each course of treatment. GCI is assessed daily and the SANS, PANNS and subset score are assessed twice a week during the course of treatment. GCI and PANNS are assessed twice a week during wash out periods. Cognitive, ERP, and clinical symptoms are assessed at week 1, 2, 4 and 8-post treatment.

Vital signs, full chemistry and hematological testing, EKG, urine analysis are performed as follows:

(1) Acute phase: during and after infusion: 0, 15, 30, 45, 60, 75, 90, 120 minutes for A-86929 plasmatic levels.

(2) Main study: the same time periods as the acute phase. For clinical analysis, all subjects are analyzed to determine whether there is cognitive function improvement and other positive clinical outcomes. Electrophysiological correlates also are evaluated.

### Example 4

### The Effect of DAS-431 on Cognitive Function Based on the COMT Genotype of the Normal or Schizophrenic Subject

The purpose of this study is to evaluate the efficacy of DAS-431 in COMT genotyped normal volunteers and schizophrenic subjects. The objectives are as follows:

(1) To select a pharmacologically active and safe dose on brain activity assessed by fMRI in genotyped normal volunteers.

(2) To explore genetic variations in response to DAS-431 on multiple cognitive tasks and to assess the relationship between individuals with the COMT Val/Val, the COMT Met/Met and the COMT Val/Met genotype.

(3) To map the neurophysiological correlates of these effects using fMRI combined with the performance of parametric variation of the N-back working memory test and the Stemberg paradigm of DAS-431.

(4) To assess the safety of five day treatment of daily IV infusion.

This is a three successive step phase IIa study. (a) An initial randomized, double blind, placebo controlled, within subject, dose finding study of the effect of single doses of DAS-431 on brain activity in genotyped normal volunteers followed by (b) a randomized, double blind, cross-over, placebo controlled study of the effects of one pharmacological active dose of DAS-431 on cognitive function and neuroimaging correlates (fMRI) in COMT genotyped normal subjects, followed by (c) a randomized, double blind, cross-over, placebo controlled study of the effects of one pharmacological active dose of DAS-431 on cognitive function and neuroimaging correlates (fMRI) in COMT genotyped schizophrenic subjects.

(1) N=6 COMT genotyped normal volunteers; depending on the observation, another 6 are enrolled.

(2) 90 COMT genotyped normal volunteers total
(a) Val/Val: total 30 subjects
(b) Val/Met: total 30 subjects
(c) Met/Met: total 30 subjects

(3) 90 COMT genotyped schizophrenic subjects total
(a) Val/Val: total 30 subjects
(b) Val/Met: total 30 subjects
(c) Met/Met: total 30 subjects

Healthy volunteers are eligible to enroll if they are COMT genotyped; meet the criteria for the NIH protocol 95-M-0150; and have no Axis I or Axis II diagnosis. Schizophrenic subjects are eligible to enroll if they meet DSM IV criteria for schizophrenia and are receiving long-term and stable antipsychotic medications; and are COMT genotyped.

The following is the treatment regimen for each of the three phases of the study:

(1) DAS-431 single daily IV 30 minute infusion, at 0.25, 0.5, 1 and 2 mg

(2) DAS-431 multiple IV 30 minute infusion for five consecutive days, at the pharmacologically dose based on fMRI performed (1)

(3) DAS-431 multiple IV 30 minute infusion for five consecutive days, at a fixed dose derived from data of study (2)

### Placebo is administered as the test drug, in each of the three phases of the study

The efficacy of the treatment regimens is measured in healthy volunteers by fMRI signals. In schizophrenic subjects, efficacy is measured by neuropsychological assessment: two batteries of tests assessing prefrontal, entorinal/hyppocampal, premotor and occipito-parietal lobe function; neuroimaging by fMRI, under performance of the N-back memory test and the Stenberg paradigm; and clinical outcomes. Vital signs, full chemistry and hematological testing, EKG, urine analysis in phase (1) are performed during and after infusion: 0, 15, 30, 45, 60, 75, 90, 120 minutes for A-86929 plasmatic levels. In phases (2) and (3): the same analysis times are used as for phase (1). Statistical analysis of phase (1) fMRI measurements is signal to noise, and variance in image standard deviation in Voxel intensities over time. In phases (1), (2), (3) statistical analysis is performed for the correlation of reaction times and performance scores with Bold signal change and for the neuropsychological data ANOVAS, with time as repeated measure and order and genotype as main effects.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

1. A method for treating cognitive impairment, the method comprising a step of:
providing to a patient in need thereof an amount of one or more selective dopamine D1-like receptor agonists effective for treating the cognitive impairment using a dosing regimen that minimizes side effects and/or desensitization or tolerance to the treatment and/or provides for long term improvement in cognitive function and/or prevention of further deterioration of cognitive function.

2. The method of claim 1, further comprising a step of:
improving other symptoms related to an underlying disease or cause of the cognitive impairment.

3. The method of claim 1, further comprising a step of:
combining the selective dopamine D₁-like receptor agonist with a second drug having some efficacy for treating an underlying disease or cause of the cognitive impairment.

4. The method of claim 3, wherein the second drug is a selective dopamine D2 receptor agonist.

5. The method of claim 4, wherein the selective dopamine D2 receptor agonist is haloperidol.

6. The method of claim 3, wherein the combination of the selective dopamine D1-like receptor agonist and the second drug acts additively or synergistically.

7. The method of claim 1, wherein the selective dopamine D1-like receptor agonist is DAS-431.

8. A method for generating a profile for correlating dopaminergic state to cognitive impairment, the method comprising steps of:
determining a dopaminergic status and/or cognitive function of both individual patients with cognitive impairment and of a population of patients with cognitive impairment associated with a particular disease state or normal aging process; and
correlating the dopaminergic status and/or the cognitive function both in the individual patient and in the particular population of patients.

9-12. (canceled)

13. A pharmaceutical pack or kit for the treatment of cognitive impairment comprising one or more selective dopamine D1-like receptor agonist

14. A pharmaceutical pack or kit for the treatment of cognitive impairment comprising one or more selective dopamine D1-like receptor agonist and a selective dopamine D2 receptor agonist.

15. The pharmaceutical pack or kit according to claim 13 or claim 14, wherein said dopamine D1-like receptor agonist comprises DAS-431.

16. The pharmaceutical pack or kit according to claim 14 or claim 15, wherein said selective dopamine D2 receptor agonist comprises haloperidol.

17. A composition comprising one or more selective dopamine D1-like receptor agonist and a pharmaceutically acceptable carrier or excipient.

18. A composition comprising one or more selective dopamine D1-like receptor agonist and a selective dopamine D2 receptor agonist in a pharmaceutically acceptable carrier or excipient.

19. The composition according to claim 17 or claim 18, wherein said selective dopamine D1-like receptor agonist comprises DAS-431.

20. The composition according to claim 18 or claim 19, wherein said selective dopamine D2 receptor agonist comprises haloperidol.

21-28. (canceled)
